(19) United States
(12) Patent Application Publication
(10) Pub. No.: US 2008/0167545 A1
(43) Pub. Date: Jul. 10, 2008

(54) Clinical workflow for combined 2D/3D diagnostic and therapeutic phlebograph examinations using a robotic angiography system

(76) Inventors: Oliver Meissner, Munchen (DE); Michael Maschke, Lonnerstadt (DE)

Correspondence Address: BRINKS HOFER GILSON & LIONE, P.O. BOX 10395, CHICAGO, IL, 60610, US

(21) Appl. No.: 11/651,246

(22) Filed: Jan. 9, 2007

(51) Int. Cl. A61B 5/05

(52) U.S. Cl. 600/407

(57) ABSTRACT

A system for performing phlebographic examinations is disclosed. In one embodiment, the system includes a patient support apparatus, a medical imaging device, and a processor coupled with the medical imaging device. The patient support apparatus is operative to support a patient in a resting position. The medical imaging device is operative to acquire an image of a portion, such as the pulmonary arteries, of the patient during a phlebographic examination. The medical imaging device may acquire the image using a two-dimensional imaging technique, a three-dimensional imaging technique, or combinations thereof. The processor coupled with the medical imaging device is operative to receive the acquired image of the patient to generate a visualization of the image of the portion of the patient. A display device coupled with the processor displays the two-dimensional or three-dimensional visualization.

### BACKGROUND

1. Technical Field

The present embodiments relate to a system and method for assisting in the diagnosis of phlebographic examinations. In particular, phlebographic examinations using a robotic angiography suite supporting multiple imaging modalities.

2. Related Art

Phlebographic examinations are examinations performed by a doctor or other medical personnel to determine the existence of a deep vein thrombosis or pulmonary embolism of a patient. In general, phlebography (also called venography, ascending contrast phlebography, or contrast phlebography) is an invasive diagnostic test that provides angiographic images of the venous vessels of the upper and lower legs, the vena cava (cavography), the pulmonary arteries (pulmonary angiography), or combinations thereof. Phlebography identifies the location and extent of blood clots, and enables the condition of the deep leg veins to be assessed. It is especially useful when there is a strong suspicion of deep vein thrombosis, after noninvasive tests have failed to identify the disease.

In today's imaging systems, duplex ultrasound has become a preferred diagnostic modality for the diagnosis of deep vein thrombosis or venous insufficiency with regard to the superficial and deep venous system of the lower or upper extremities. In addition to duplex ultrasound, venography may be used for patients compromised by recent surgery or injury involving the affected limb, confusing anatomy, adipositas, and those patients with known lesions that require further delineation before treatment. Indications for the use of venography include the delineation of deep vein thrombosis (DVT) as a prelude to a catheter-directed intervention such as a thrombolysis or a thrombectomy, the presence of a nondiagnostic or technically poor ultrasound, or the need to obtain a more detailed image of the calf veins.

In the diagnosis of a pulmonary embolism, doctors or clinicians may use computed tomography as an imaging modality. Alternatively, or in addition to computed tomography, magnetic resonance imaging may be used for some patients. However, doctors or clinicians may not use computed tomography or magnetic resonance for direct interventions, such as catheter-based interventions. For example, where computed tomography angiography or magnetic resonance angiography has yielded inconclusive or otherwise non-diagnostic results, or when negative studies conflict with a strong clinical suspicion for pulmonary embolism, catheter-based pulmonary arteriography, may be used alternatively to, or in addition to, these methods. In general, traditional pulmonary angiography is used for the identification of small or subtle abnormalities such as peripheral or chronic emboli, and for detection of emboli in vessels oriented along the axial plane, which may be missed by computed tomography angiography or magnetic resonance angiography. In addition, a pulmonary arterial catheter can provide direct measurement of pulmonary arterial pressure.

In contemporary practice, the diagnosis of patients suspected of having a pulmonary embolism without deep vein thrombosis is complex and time-consuming. In general, the treatment of a pulmonary embolism first includes performing a D-dimer test on the patient in the emergency room. D-dimer tests are often ordered, along with other laboratory tests and imaging scans, to help rule out, diagnose, and monitor diseases and conditions that cause hyper-coagulability, a tendency to clot inappropriately. One of the most common of these conditions is deep vein thrombosis, which involves clot formation in the deep veins of the body, most frequently in the legs. These clots may grow very large and block blood flow in the legs, causing swelling, pain, and tissue damage. It is possible for a piece of the clot to break off and travel to other parts of the body, where the clot can cause a pulmonary embolism.

Following the D-dimer test in traditional diagnosis, the patient then often undergoes several different types of imaging, including, transferring the patient to an ultrasound department to perform a duplex sonography; transferring the patient to a computed tomography department to perform a computed tomography scan, a magnetic resonance scan, or combinations thereof, of the thorax to rule out or confirm a pulmonary embolism; transferring the patient to an angiographic suite to perform radiological intervention; and, finally transferring the patient back to the emergency room. Aside from the fact that the entire process can be time-consuming, each scan of the patient can be complex and time-consuming as well.

Throughout the diagnosis process, each aforementioned imaging system may have insufficient patient access for this complex procedure to be individually effective. Furthermore, these imaging systems often lack the ability to perform imaging scans of the patient's peripheral venous and pulmonary arteries in one session. Lastly, the difficulty in exactly planning contrast delivery coupled with the limited degree of movement of the imaging equipment, hinders the diagnosis and therapy of deep vein thrombosis, pulmonary embolism, or combinations thereof.

### BRIEF SUMMARY

By way of introduction, the embodiments described below include a system and a method for performing phlebographic examinations. In one embodiment, the system includes a patient support apparatus, a medical imaging device, and a processor coupled with the medical imaging device. The patient support apparatus is operative to support a patient in a resting position. The medical imaging device is operative to acquire an image of a portion of the patient during a phlebographic examination. The medical imaging device may acquire the image using one of at least two imaging modalities. The two imagining modalities may include one or more two-dimensional imaging modalities, one or more a three-dimensional imaging techniques, or combinations thereof. The processor coupled with the medical imaging device is operative to receive the acquired image of the patient to generate a visualization of the image. The visualization of the portion of the patient may be displayed on a display device coupled with the processor.

In one embodiment, the method for performing the phlebographic examination includes receiving a patient from a treatment location and transferring the patient to the patient support apparatus. The method also includes determining at least one phelographic examination, and performing the phlebographic examination on the patient using the medical imaging device. The method further includes returning the patient to the treatment location. In an alternative embodiment, the method includes determining whether to perform a therapeutic treatment on the patient, performing the therapeutic treatment on the patient, and then verifying whether the therapeutic treatment was successful. In yet another embodiment, the method includes determining whether the patient has a perceived difficulty in breathing, and adjusting the patient support apparatus to support the patient in a position that alleviates the difficulty the patient has in breathing.

The present invention is defined by the following claims, and nothing in this section should be taken as a limitation on those claims. Further aspects and advantages of the embodiments are discussed below in conjunction with the preferred embodiments and may be later claimed independently or in combination.

### BRIEF DESCRIPTION OF THE DRAWINGS

FIG. 1 is a block diagram of one embodiment of a system for performing phlebographic examinations.

FIG. 2A is a schematic diagram of one embodiment of a robotic arm with six axes of rotation according to the prior art.

FIG. 2B is a schematic diagram of one embodiment of a medical imaging device for performing phlebographic examinations.

FIG. 3A is a perspective view of one embodiment of the medical imaging device and a patient support apparatus for performing phlebographic examinations.

FIG. 3B is an alternative perspective view of the medical imaging device and the patient support apparatus for performing phlebographic examinations.

FIG. 4A is an illustration of an image captured of a patient using one modality of the medical imaging device.

FIG. 4B is an illustration of an image captured of a patient using a modality of the medical imaging device.

FIG. 4C is an illustration of yet another image captured of a patient using a modality of the medical imaging device.

FIG. 5 is a block diagram of one embodiment of the method for performing phlebographic examinations using the medical imaging device.

### DETAILED DESCRIPTION

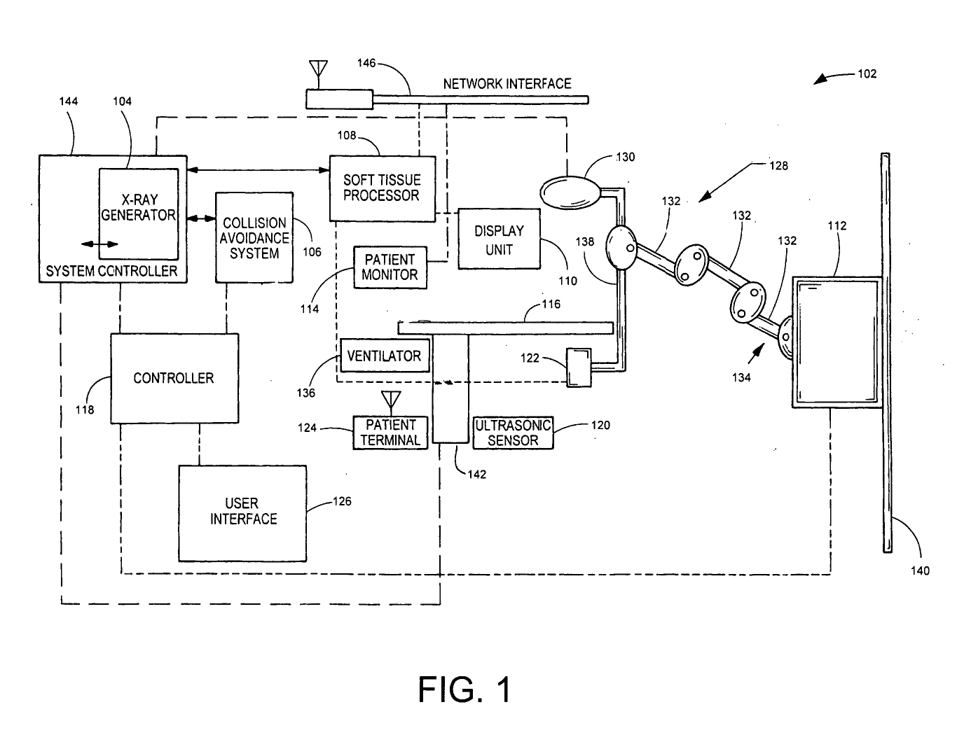

FIG. 1 is a block diagram of one embodiment of a robotic angiography suite for performing phlebographic examinations. The robotic angiograph suite shown in FIG. 1 is a robotic angiography system 102. As shown in FIG. 1, the system 102 includes a first embodiment of a medical imaging device 128, a patient support apparatus 116, and related diagnostic and treatment devices. The system 102 may include additional or fewer diagnostic and treatment devices than those shown in FIG. 1. The individual units, devices, and equipment, may communicate with each other using wired connections, wireless connections, or combinations thereof. Wired connections include, but are not limited to, PS/2, USB, Ethernet, IDE/ATA, SCSI, SATA, IEEE 1394, VGA, DVI, any other now known or later developed wired connection, or combinations thereof. Wireless connections include, but are not limited to, 802.11a/b/g, Bluetooth, RF, infrared, any other now known or later developed wireless connection, or combinations thereof. The use of dashed lines shown in FIG. 1 illustrate that alternative connections may be used for connecting one or more devices. A network interface 146 coupled with the system 102 is used to facilitate communication between the equipment and devices in the system 102. The network interface 146 is further operative to communicate with other networks, treatment systems, hospitals, clinicians, or combinations thereof, to obtain or send information regarding the phlebographic examination using the system 102. The network interface 146 may use one or more wired connections, wireless connections, or combinations thereof to facilitate communication.

The medical imaging device 128 is a medical imaging device operative to generate two-dimensional images, such as fluoroscopic images, angiographic images, ultrasound images, X-ray images, any other now known or later developed two-dimensional image acquisition technique, or combinations thereof. For example, in one embodiment the medical imaging device 128 is an X-ray imaging device, such as the ARCADIS Orbic C-arm imaging device available from Siemens Medical Solutions of Siemens AG headquartered in Malvern, Pa. In another embodiment, the medical imaging device 128 is an imaging device capable of producing fluoroscopic images, such as the AXIOM Iconos R200 also available from Siemens Medical Solutions of Siemens AG. The medical imaging device 128 could also be in an imaging device capable of producing angiographic images, such as the AXIOM Artis dTA also available from Siemens Medical Solutions of Siemens AG.

In one embodiment, the medical imaging device 128 is a C-arm X-ray apparatus 128. The C-arm X-ray apparatus 128 includes a C-arm support 138 to which an X-ray source 130 and an X-ray detector 122 are mounted. The X-ray source 130 and the X-ray detector 122 may be further mounted such that they are diametrically opposed and facing each other along a central axis of radiation or rotation. The X-ray source 130, the X-ray detector 122, or combinations thereof, may further include a diaphragm to limit the radiation field to which a patient may be exposed.

The C-arm support 138 is mounted to a robotic device 134, which is controllable by a controller 144. In one embodiment, the robotic device 134 includes articulated arms 132 that are rotatable around one or more axes. For example, and with reference to a Cartesian coordinate system, the articulated arms 132 may be rotatable around an x-axis, a y-axis, a z-axis, or combinations thereof. The controller 144 may send commands causing a motive device 112 to move the articulated arms 132. The motive device 112 may be a motor, hydraulic mechanism, any other now known or later developed motive device, or combinations thereof. In one embodiment, the motive device 112 is mounted to a wall 140. However, the motive device 112 may be mounted to a ceiling, a floor, any other now known or later developed mounting surface, or combinations thereof. The motive device 112 may be further capable of moving in a longitudinal and/or transverse direction with respect to the mounting surface 140.

The C-arm X-ray apparatus 128 is rotatable to acquire a plurality of phlebographic two-dimensional images. In general, a two-dimensional image acquired by the C-arm X-ray apparatus 128 is a fluoroscopic image, an angiographic image, an X-ray image, any other equivalent two-dimensional image, or combination thereof.

Other modalities than X-ray may be used. For example, the two-dimensional image may be acquired using ultrasound imaging, computed tomography [symbol] magnetic resonance tomography (MRT), any other two-dimensional image technique now known or later developed, or combinations thereof. In one embodiment, the two-dimensional image is acquired using computed tomography pulmonary angiography. The two-dimensional image could also be acquired using catheter venography, where a contrast agent is injected into the peripheral or deep veins of a patient followed by an X-ray scan of the patient to determine the presence of a venous thrombosis. Alternatively, the two-dimensional image could be a two-dimensional image of a scanned organ cavity or a portion of the patient undergoing the phlebographic examination. For example, the two-dimensional image may be an angiographic image of the patient's chest cavity. As another example, the two-dimensional image may be a fluoroscopic image of the patient's lower extremities. In yet a further example, the two-dimensional image may be an angiographic image of the patient's pelvis. The two-dimensional image could also be an angiographic image of the patient's inferior vena cava or abdomen, pulmonary arteries or thorax, or combinations thereof.

The C-arm X-ray apparatus 128 or other apparatus may be further operative to acquire three-dimensional image datasets used to generate three-dimensional images. In general, a three-dimensional image dataset is a dataset representative of an organ cavity or portion of the patient acquired by the C-arm X-ray apparatus 128. The three-dimensional image dataset may be acquired using any three dimensional technique, including pre-operative techniques, intra-operative techniques, or combinations thereof. Examples of pre-operative techniques include, but are not limited to, ultrasound imaging, computed tomography, traditional angiography, or combinations thereof. Examples of intra-operative techniques include, but are not limited to, 3D digital subtraction angiography, 3D digital angiography, rotational angiography, such as the Dyna-CT technique developed by Siemens Medical Solutions of Siemens AG, 3D ultrasound, or combinations thereof. Other types of three-dimensional imaging techniques known or later developed are also contemplated.

Referring further to FIG. 1, a patient support apparatus 116 is positioned near or coupled with the medical imaging device 128, such as the C-arm X-ray apparatus 128. The patient support apparatus 116 is operative to support a patient (not shown). The patient support apparatus 116 may be a stretcher, gurney, any other now known or later developed support apparatus, or combinations thereof. The patient support apparatus 116 may be mounted to a mounting surface, such as the floor, but may also be mounted to a wall, a ceiling, any other now known or later developed mounting surface, or combinations thereof.

In one embodiment, the patient support apparatus 116 is coupled with a motive device 142, which is further coupled with a system controller 144, a controller 118, or combinations thereof. The motive device 142 enables rotation and/or translation of the patient support apparatus 116. For example, the patient support apparatus 116 may be operative to rotate about one or more axes of rotation, such as being operative to rotate in a longitudinal direction about a horizontal axis, operative to rotate in a transverse direction about a vertical axis, or combinations thereof. In one embodiment, the patient support apparatus 116 is further operative to rotate about an axis of the medical imaging device 128. The patient support apparatus may further appear transparent in the acquired two-dimensional images or three-dimensional visualizations when the patient support apparatus is exposed to radiation emitted from the medical imaging device 128.

In another embodiment, the patient support apparatus 116 and the motive device 142 are coupled to form a robotic device. In this embodiment, where the patient support apparatus 116 and the motive device 142 are coupled to form a robotic device, the patient support apparatus 116 and the motive device 142 may be arranged substantially similar to the C-arm X-ray apparatus 128. For example, the patient support apparatus 116 may be mounted to one limb of a C-arm X-ray apparatus such that the patient support apparatus 116 is operative to tilt or rotate about one or more axes. Further in this embodiment, the patient support apparatus 116 and the motive device 142 may receive one or more commands from the system controller 144, the controller 118, the user interface 126, or combinations thereof.

A soft tissue processor 108 is coupled with the C-arm X-ray apparatus 128. The soft tissue processor 108 is operative to generate a two-dimensional image or visualization of the image acquired of the patient using the C-arm X-ray apparatus 128. The soft tissue processor 108 may be further operative to generate a three-dimensional visualization of the image acquired using the C-arm X-ray apparatus 128.

The soft tissue processor 108 may be a general processor, a data signal processor, graphics card, graphics chip, personal computer, motherboard, memories, buffers, scan converters, filters, interpolators, field programmable gate array, application-specific integrated circuit, analog circuits, digital circuits, combinations thereof, or any other now known or later developed processor. The soft tissue processor 108 includes software and/or hardware for displaying a two-dimensional visualization of the portion of the patient acquired using the C-arm X-ray apparatus 128, such as by displaying pixels of the two-dimensional visualization according to relative radiodensity. The soft tissue processor 108 also includes software or hardware for rendering a three-dimensional representation of the three dimensional image dataset, such as through alpha blending, minimum intensity projection, maximum intensity production, surface rendering, any other now known or later developed rendering technique, or combinations thereof.

A display unit 110 is coupled with the soft tissue processor 108. The display unit 110 may be further coupled with other equipment or devices shown in FIG. 1, such as the patient monitor 114, the ventilator 136, a patient terminal 124, an ultrasonic sensor 120, or combinations thereof. The display unit 110 is a monitor, CRT, LCD, plasma screen, flat-panel, projector, any other now known or later developed display device, or combinations thereof. The display device unit 110 is operable to display the two-dimensional visualization of the imaged portion of the patient, the three-dimensional visualization of the imaged portion of the patient, or combinations thereof, generated by the soft tissue processor 108. The display unit 110 may be further operable to display a separate three-dimensional image of the imaged portion of the patient and a display of the two-dimensional visualization of the imaged portion of the patient captured by the C-arm X-ray apparatus 128. The display unit 110 can also be configured to display information relating to the statistics and information stored or monitored by the ventilator 136, the ultrasonic sensor 120, a collision avoidance system 106, a user interface 126, the patient terminal 124, or combinations thereof.

The system 102 may further include an ultrasonic sensor 120. The ultrasonic sensor 120 may be coupled to the C-arm X-ray device 128, the patient support apparatus 116, any other devices or equipment in the system 102, or combinations thereof. In one embodiment, the ultrasonic sensor 120 is operative to measure the relative distance between parts of the C-arm X-ray device 128, the patient support apparatus 116, any other devices or equipment in the system once or two, or combination thereof, so as to aid in avoiding undesired contact between the devices or equipment. The ultrasonic sensor 120 produces relative position data and ultrasonic data, which may be communicated to the collision avoidance system 106. The collision avoidance system 106 may be configured to prevent unsafe positioning. The ultrasonic sensor 120 may be further configured to supplement other determinations of relative position with respect to sensors or controls in each of the devices or equipment of the system 102. In one embodiment, the ultrasonic sensor 120 is used as a positioning input to prevent devices or equipment in the system 102 from entering within range of a predetermined distance from each other.

In addition, or alternatively to being a positioning input, the ultrasonic sensor 120 may be configured to perform ultrasound examinations, such as a duplex sonography. In general, duplex sonography refers to the use of Doppler and conventional ultrasound to allow a physician to view the structure of blood vessels. Duplex sonography may produce images that can be color coded to show a physician where a patient's blood flow is severely blocked as well as the speed and direction of the blood flow. In one embodiment, the ultrasonic sensor 120 comprises a transducer used to emit and receive sound waves. Whether the ultrasonic sensor 120 comprises a transducer used to scan a portion of a patient in an ultrasound examination, the soft tissue processor 108 may be configured to process the received waves to reconstruct a two-dimensional image, a three-dimensional image, or combinations thereof, of the portion of the patient that was scanned.

In one embodiment, the C-arm X-ray apparatus 128 is controlled by a system controller 144. The system controller 144 may further include an X-ray generator 104, a high-voltage power supply, or combinations thereof. The phlebographic examination system 102 may also include a patient monitor 114, a ventilator 136, a patient terminal 124, and the user interface 126.

The patient monitor 114 may be coupled with the patient support apparatus 116 to monitor the vital signs of the patient undergoing the phlebographic examination. In one embodiment, the patient monitor 114 is an electrocardiogram. The patient monitor 114 may be further coupled to the soft tissue processor 108 for producing two-dimensional and three-dimensional visualizations of the imaged portion of the patient over a period of time. For example, the patient monitor 114 and the soft tissue processor 108 may be used in conjunction to produce four-dimensional visualizations of an imaged portion of the patient, represented by as a two-dimensional visualization, a three-dimensional visualization, or combinations thereof, changing over a period of time.

The patient terminal 124 is operative communicate information about the patient to and from the robotic angiographic system 102. For example, the patient terminal 124 may use a wired connection, a wireless connection, or combinations thereof, to communicate the information about the patient to and from the phlebographic examination system 102. Patient information may include, but is not limited to, demographic information, prescription information, prior treatment information, prior ailments, current status conditions, current health information, or combinations thereof. For example, a user of the phlebographic examination system 102 could use the patient terminal 124 to determine whether the patient undergoing the phlebographic examination has suffered from a pulmonary embolism in the past, or has been previously diagnosed with deep vein thrombosis.

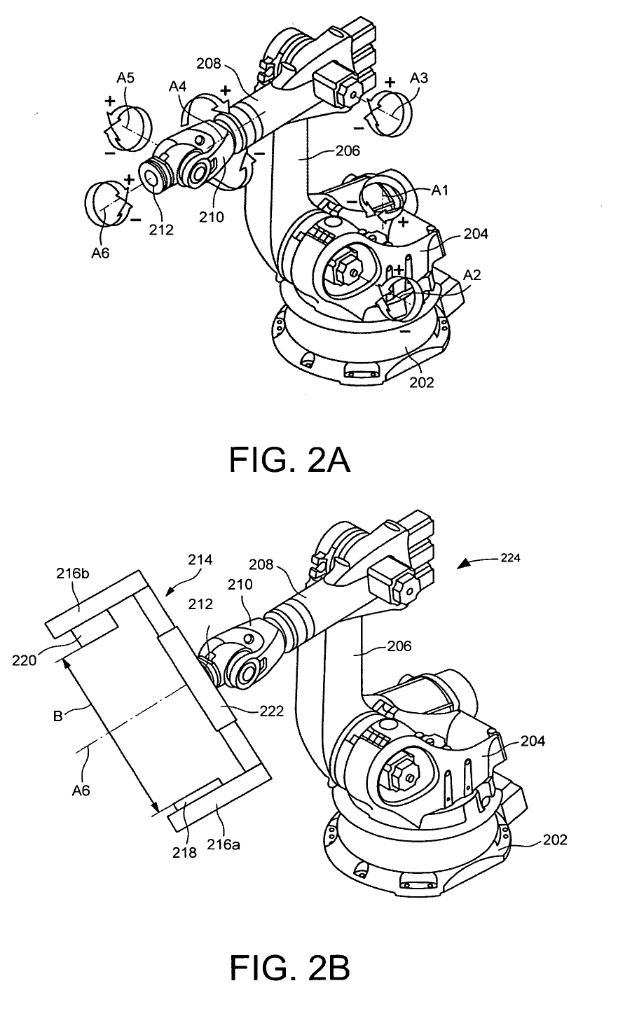

FIG. 2A is a schematic diagram of one embodiment of a robotic arm with six axes of rotation according to the prior art. A turntable 204 is mounted on a base frame 202, which is installed permanently on a mounting surface, such as a floor. The turntable 204 is operative to rotate about a first axis of rotation A1. A floating link 206 is attached to the turntable 204 so as to be capable of swiveling about a second axis of rotation A2. An arm 208 is fixed to the floating link 206 so as to be capable of rotating about a third axis of rotation A3. A hand 210 is attached the end of the arm 208 so as to be capable of rotating about a fourth axis of rotation A4. The hand 210 has a fixing element 212 which is capable of rotating about rotational axis A6 and swiveling about a fifth axis of rotation A5 running perpendicular to it.

FIG. 2B is a schematic diagram of a second embodiment of a medical imaging device 224 for performing phlebographic examinations according to the system 102 of FIG. 1. The medical imaging device includes a support apparatus 214 connected to the fixing element 212. A connection not shown in detail here can be provided for connecting and disconnecting the support apparatus 214 to a fixing element 212. The support apparatus 214 includes a central member 222 extending in a perpendicular direction to the fixing element 212. Extending off either end of the central member 222 is a first limb 216a and a second limb 216b. An X-ray detector 218 is attached to the first limb 216a and an X-ray source is attached to the second limb 216b. The first limb 216a and the second limb 216b are attached to the central member 222 so as to be capable of linear movement along the central member 222. Throughout a phlebographic examination the first limb 216a and the second limb 216b may be maintained at a predetermined distance B. However, as the first limb 216a and the second limb 216b are also capable of movement along the central member 222, the predetermined distance B may be adjustable before, during, and after a phlebographic examination. The second embodiment of the medical imaging device 224 may be used alternatively to, or in addition to, the first embodiment of the medical imaging device 128 shown in FIG. 1.

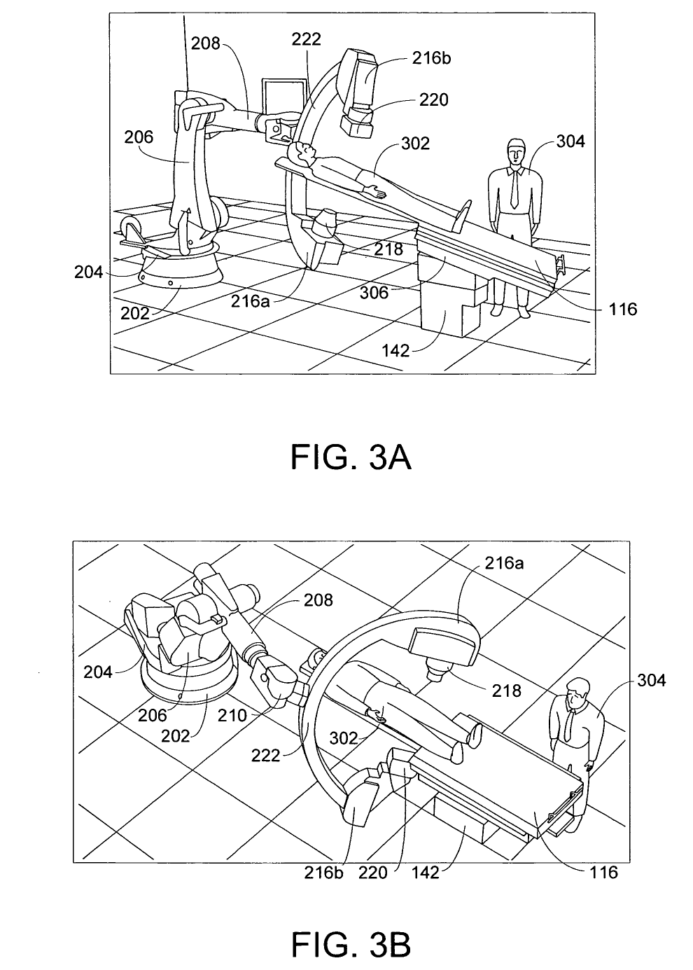

With reference to FIG. 1 and FIG. 2B, FIG. 3A is a perspective view of one embodiment of the medical imaging device 224 and a patient support apparatus 116 for performing phlebographic examinations. The patient support apparatus 116 is at an angled position such that a patient 302 lying on the patient support apparatus 116 is also at an angled position. A pivot location 306 between the motive device 142 and the patient support apparatus 116 enables the patient apparatus 116 to rotate about one or more axes. Although shown in an angled position of substantially 45°, the patient support apparatus 116 may be further operative to tilt through degrees of movement between 0° and 90°, inclusive.

With the patient 302 supported by the patient support apparatus 116, the medical imaging device 224 is able to navigate into a position substantially close to the patient 302. As shown in FIG. 3A, the medical imaging device 224 is able to navigate to the patient 302 such as to place the patient 302 between the x-ray detector 218 mounted to the first limb 216a and the x-ray source 220 mounted to the second limb 216b. Using its multiple axes of rotation, the medical imaging device 224 can then rotate about a portion or location of the patient 302 to obtain one or more of angiographic or fluoroscopic two-dimensional images, a three-dimensional image dataset, or combinations thereof. The three-dimensional image dataset can then be used to create a three-dimensional image of the scanned portion or location of the patient 302.

Furthermore, because the medical imaging device 224 is operative to operate in one or more modalities, the patient 302 can remain on the patient support apparatus 116 during one of more scans by the medical imaging device 224 throughout the phlebographic examination. For example, as an initial step of the phlebographic examination, the ultrasound sensor 120 may be first operative to perform a duplex sonography of the patient 302.

Following the duplex sonography in traditional phlebographic examinations, the patient 302 would be transferred to another location for additional scanning. However, instead of transferring the patient 302 to another location or other department, the medical imaging device 224 can then operate in a computed tomography modality to perform a computed tomography scan of the patient 302. For example, the medical imaging device 224 may perform a computed tomography scan of the patient's 302 thorax to determine whether the patient 302 suffers from a pulmonary embolism. Furthermore, the patient support apparatus 116 can be rotated about the pivot location 306 using the motive device 142 to obtain images that would otherwise require the transfer of the patient 302 to a separate facility.

Where the computed tomography scan reveals that the patient suffers from a pulmonary embolism, a traditional phlebographic examination would require that the patient 302 be transferred to a separate angiographic examination suite. However, because the medical imaging device 224 can rotate freely about the patient 302 along one or more axes, the medical imaging device 224 can be configured to perform pulmonary angiographies. In one embodiment, the robotic angiography system 102 can perform one or more angiographic examination, phlebographic examination, or combinations thereof, of the complete body of a patient. In addition, the soft tissue processor 108 can be configured to reconstruct CT-like soft tissue images from the volumetric data set of the scanned one or more portions of the patient. For example, the soft tissue processor 108 can use different imaging settings for the delineation of different tissue qualities, such as soft tissue, bone, any other delineation of tissue quality, or combinations thereof.

Using the robotic angiography system 102, a doctor 304 can perform a radiological intervention on the patient 302 to remove or treat the pulmonary embolism. Due to the arrangement of the medical imaging device 224 in relation to the patient support apparatus 116, the doctor 304 is able to position himself closer to the patient 302 than in traditional phlebographic examinations. Hence, the doctor 304 has better access and readier availability to the patient 302 than in traditional phlebographic examinations. Following the interventional procedure, the doctor 304 may then perform additional scanning of the patient 302 using the medical imaging device 224 to determine whether the interventional procedure was successful.

With reference to FIG. 1 and FIG. 2B, FIG. 3B is an alternative perspective view of the medical imaging device 224 and the patient support apparatus 116 for performing phlebographic examinations. In the view shown in FIG. 3B, the patient support apparatus 116 is arranged in a substantially horizontal position, such that the patient is substantially parallel to the floor to which the patient support apparatus 116 is mounted. Similarly to the phlebographic examination described with reference to FIG. 3A, the doctor 304 can perform a phlebographic examination on the patient 302 with the patient 302 lying in this horizontal position. Although not shown, the doctor 304 can also perform phlebographic examinations where the patient support apparatus 116 is substantially vertical such that the patient 302 is substantially perpendicular to the floor where the patient support apparatus 116 is mounted.

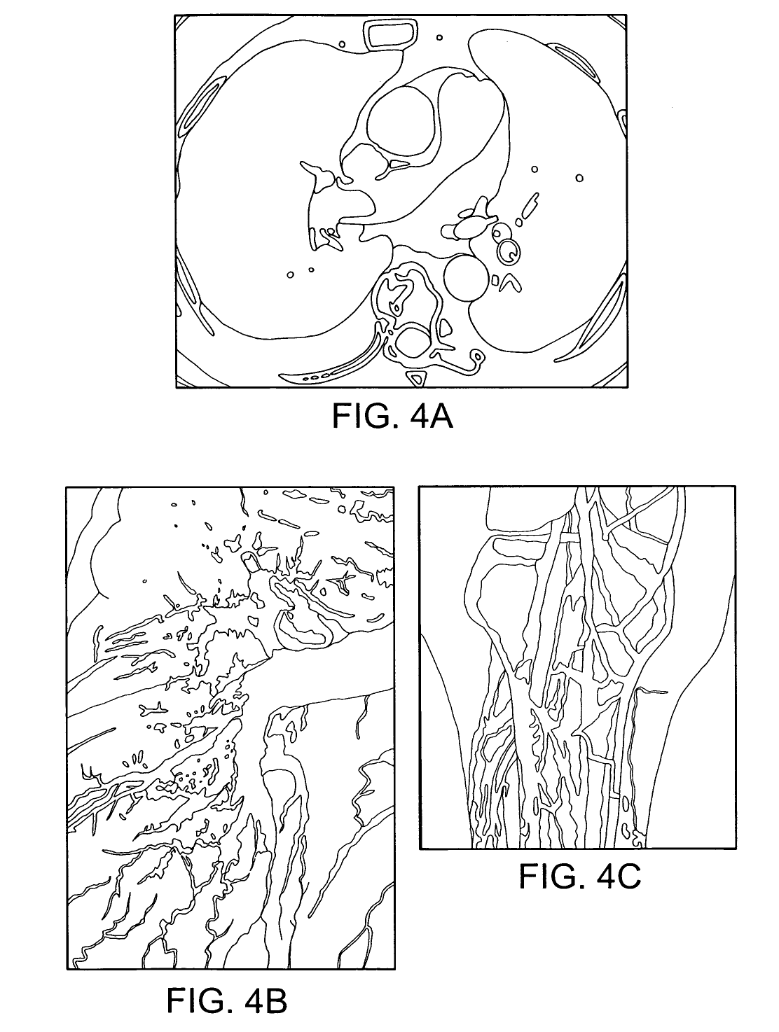

With reference to FIG. 1 and FIG. 2B, FIG. 4A, FIG. 4B, and FIG. 4C, are visualizations of different organ cavities of the patient 302 captured using the medical imaging device 224 during the phlebographic examination of the patient 302. FIG. 4A is a an axial slice of a computed tomography angiography showing a large thrombus formation/embolus (dark portion in the bright, contrast filled vessel) in the right main pulmonary artery as well as in large pulmonary arteries on the left. FIG. 4B is a two-dimensional image captured by the medical imaging device 224 using traditional pulmonary angiography as an imaging modality. In particular, FIG. 4B depicts large thrombus formations in pulmonary arteries on the right side of the image, represented by filling defects. FIG. 4C is a two-dimensional image captured by the medical imaging device 224 using catheter venography depicting venous thrombosis of the lower extremity of the patient 302. In general, catheter venography involves the use of a contrast agent and x-rays to determine the possibility of thrombosis, and FIG. 4C depicts extensive thrombosis of the tibial and popliteal veins manifested as luminal filling defects surrounded by the contrast.

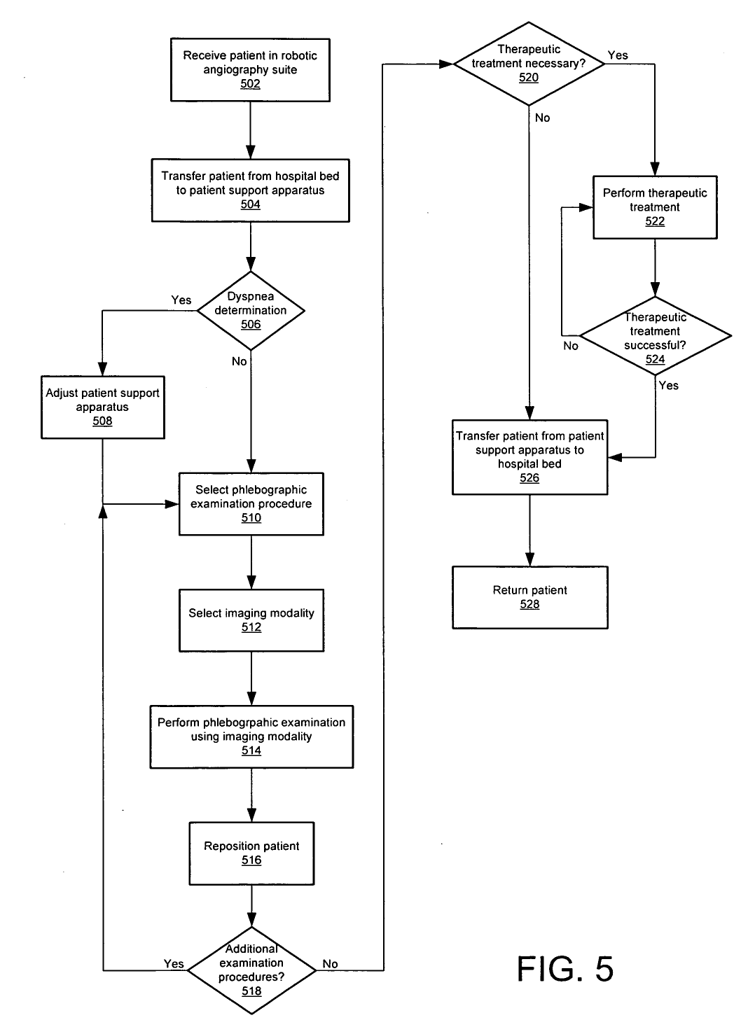

Turning now to FIG. 5 with reference to FIG. 1 and FIG. 2B, is a block diagram of one embodiment of the method for performing phlebographic examinations using the medical imaging device 224 and the patient support apparatus 116. Additional, fewer, and/or different acts may be performed than shown in FIG. 5.

As an initial matter, a patient 302 is transferred to a robotic angiography suite (Block 502). The robotic angiographic suite includes the medical imaging device 128, the patient support apparatus 116, and one or more of the equipment or devices shown in FIG. 1. The robotic angiographic suite may also include the medical imaging device 224 in addition to, or alternatively to, the medical imaging device 128 shown in FIG. 1. After receiving the patient 302 in the robotic angiographic suite (Block 502), the patient 302 is then transferred from a hospital bed to the patient support apparatus 116 (Block 504). In one embodiment, the patient support apparatus 116 is configured to help and transfer the patient 302 from the hospital bed to the patient support apparatus 116. For example, the patient support apparatus 116 may be operative to rotate about a longitudinal axis using the motive device 142, in which case, the patient support apparatus 116 can rotate to meet the hospital bed and be used to transfer the patient 302 to the patient support apparatus 116. Alternatively, the patient 302 may use other means, such as walking, to approach the and enter the patient support apparatus 116.

After the patient 302 has been transferred to the patient support apparatus 116, a doctor 304 or other medical personnel can make a determination whether the patient 302 suffers from dyspnea (Block 506). In general, dyspnea is the perceived difficulty in breathing or pain on breathing. If the doctor 304 or other medical personnel determines that the patient 302 suffers from dyspnea, the doctor 304 can adjust the patient support apparatus 316 to accommodate the patient 302 (Block 508). In one embodiment, where the patient 302 suffers from dyspnea while recumbent, the patient support apparatus 316 is rotatable about a lateral axis such that the patient support apparatus 316 can be rotated to an upright or vertical position. In another embodiment, where the patient 302 suffers from dyspnea while upright, the patient support apparatus 316 is rotatable so that it can be placed in a substantially horizontal position. The patient support apparatus 316 may be further rotatable about additional axes to further accommodate the needs of the patient 302, the doctor 304, additional equipment in the robotic angiography suite, or combinations thereof.

After the patient support apparatus 316 has been adjusted (Block 508) or if the doctor 304 has determined that the patient 302 does not suffer from dyspnea (Block 506), the doctor 304 then selects a phlebographic examination procedure (Block 510). As discussed previously, phlebographic examination procedures include, but are not limited to, a phlebography of the lower extremities, cross-sectional imagery of the pelvis, a phlebography of the inferior vena cava or abdomen, and a pulmonary angiography or cross-sectional imagery of the pulmonary arteries or thorax. The term phlebography includes venography, ascending contrast phlebography, contrast phlebography, and combinations thereof. For example, the doctor 304 may select a phlebography of the lower extremities of the patient 302 to determine whether the patient suffers from deep vein thrombosis. Alternatively, the doctor 304 could select to perform a pulmonary angiography of the patient 302 to determine whether the patient 302 suffers from a pulmonary embolism. As yet another example, the doctor 304 could select to perform a catheter-based venography to determine whether the patient 302 suffers from thrombosis.

In one embodiment of the robotic angiography suite, the soft tissue processor 108, or other processor, may be preprogrammed to display a list of phlebographic examinations on the display unit 110. The doctor 304 could then use the user interface 126 to select one or more phlebographic examinations displayed by the display unit 110. Alternatively, or in addition to, the list of phlebographic examinations displayed by the display unit 110, the doctor 304 could use the user interface 126 to manually select or program a phlebographic examination for the soft tissue processor 108.

After selecting the phlebographic examination procedure (Block 510), an imaging modality is then selected to perform the phlebographic examination procedure (Block 512). As previously discussed, the first embodiment of the medical imaging device 128 or the second embodiment of the medical imaging device 224 is configured to perform one or more imaging modalities. Imaging modalities performable include, but are not limited to, two-dimensional imaging techniques or three-dimensional imaging techniques. The imaging modality may be selected by the soft tissue processor 108, the doctor 304, or combinations thereof. In one embodiment, the doctor 304 chooses which imaging modality to use for the selected phlebographic examination procedure. For example, the doctor 304 may choose a two-dimensional imaging technique, such as duplex sonography, traditional pulmonary angiography, computed tomography pulmonary angiography, magnetic resonance imaging, any other now known or developed two-dimensional imaging technique, or combinations thereof. Alternatively, or in addition to the selected two-dimensional imaging technique, the doctor 304 may select a three-dimensional imaging technique, such as Dyna-CT, computed tomography, any other now known or later developed three-dimensional imaging technique, or combinations thereof.

In an alternative embodiment, the soft tissue processor is pre-configured to select the appropriate imaging modality for the selected phlebographic examination procedure. For example, where the selected phlebographic examination procedure is a phlebography of the lower extremities, the soft tissue processor 108 or other device or equipment may automatically select a two-dimensional imaging modality, such as computed tomography.

As another example, where the selected phlebographic examination procedure is a phlebography of the thorax (Block 510), the soft tissue processor 108 may select a three-dimensional imaging technique, such as Dyna-CT, as the imaging modality. The soft tissue processor 108, or other device for equipment, may select other imaging modalities including, but not limited to, traditional pulmonary angiography, computed tomography pulmonary angiography, and any other now known or later developed two-dimensional or three-dimensional imaging techniques, or combinations thereof.

After the imaging modality is selected (Block 512), the phlebographic examination is then performed on the patient 302 using the selected imaging modality (Block 514). The phlebographic examination may be performed by the doctor or other clinician 304, the soft tissue processor 108, any other devices or equipment, such as the system controller 144, or combinations thereof. The selected phlebographic examination may involve scanning of the patient 302 using the selected imaging modality. The scanning may be performed by the first embodiment of the medical imaging device 128, the second embodiment of the medical imaging device 224, or combinations thereof. For example, where the second embodiment of the medical imaging device 224 is used to scan the patient 302, the medical imaging device 224 may rotate about one or more axes to scan one or more regions of the patient 302 according to the selected phlebographic examination procedure. The results of the scan or of the phlebographic examination may be displayed on the display unit 110. For example, a two-dimensional visualization, a three-dimensional visualization, or combinations thereof, of the scan or phlebographic examination may be displayed on the display unit 110.

After the selected phlebographic examination procedure is performed on the patient 302 (Block 514), the doctor 304, the soft tissue processor 108, the motive device 142, or combinations thereof repositions the patient (Block 516). For example, where a scan of the patient 302 is required from a different angle, the motive device 142 may be used to adjust the patient support apparatus 116 about one a more axes to reposition the patient 302. As another example, the selected phlebographic examination may require multiple scans of the patient, in which case the patient 302 may be repositioned by adjusting the patient support apparatus 116. Alternatively, additional scans may not be required for the selected phlebographic examination procedure, in which case the patient is not repositioned.

Following the performance of the selected phlebographic examination procedure (Block 514), a determination is then made as to whether additional phlebographic examinations are required or have been selected (Block 518). The determination as to whether additional phlebographic examination procedures are required or have been selected may be performed by the doctor 304, the soft tissue processor 108, the system controller 144, any other device or equipment, or combinations thereof. In one embodiment, the doctor 304 is able to select multiple phlebographic examinations initially, such that the system 102 performs the phlebographic examinations in sequence until all phlebographic examinations have been performed. In another embodiment, the doctor 304 is offered a choice via the display unit 110 and user interface 126 to select additional phlebographic examination procedures. In yet a further embodiment, the soft tissue processor 108 performs additional phlebographic examination procedures according to preconfigured or preprogrammed processes. Where additional phlebographic examination procedures are to be performed, the method may return to the act of selecting a phlebographic examination procedure (Block 510), the act of selecting an imaging modality (Block 512), the act of performing the phlebographic examination procedure (Block 516), or combinations thereof.

When the phlebographic examination procedures have been completed (Block 518), a determination is then be made as to whether one or more therapeutic treatments are required (Block 520). For example, where the results of the one or more phlebographic examinations reveal that the patient 302 has a pulmonary embolism, the doctor 304, the soft tissue processor 108, other device or equipment, or combinations thereof may determine a desire or need to perform a therapeutic treatment to treat the pulmonary embolism. As another example, where the result of the one or more phlebographic examinations reveal that the patient 302 suffers from deep vein thrombosis, the doctor 304, the soft tissue processor 108, other device or equipment, or combinations thereof may determine the need or desire to perform a therapeutic treatment to treat the deep vein thrombosis. Alternatively, the doctor 304, the soft tissue processor 108, other device for equipment, or combinations thereof may determine that no therapeutic treatments are necessary.

Once a determination has been made that one or more therapeutic treatments are desired or necessary, the doctor 304, the soft tissue processor 108, or combinations thereof performs a therapeutic treatment (Block 522). As previously discussed above, the therapeutic treatments may be related to treating a pulmonary embolism or deep vein thrombosis. A performed therapeutic treatment may include, but is not limited to, a catheter-based treatment, thrombolytic therapy, anticoagulation therapy, any other now known or later developed therapeutic treatment for treating deep vein thrombosis or pulmonary embolism, or combinations thereof. Thrombolytic agents include, but are not limited to, streptokinase, urokinase, recombinant tissue plasminogen activator, heparin, warfarin, any other now known or later developed thrombolytic agents, or combinations thereof.

After performing a therapeutic treatments on the patient 302 (Block 522), the doctor 304, the soft tissue processor 108, or combinations thereof then verifies whether the therapeutic treatment was successful (Block 524). Verification of whether the therapeutic treatment was successful may include using the medical imaging device 128, the medical imaging device 224, any other device or equipment of the system 102, or combinations thereof. Verification could also include the use of one or more imaging modalities, such as computed tomography pulmonary angiography, magnetic resonance pulmonary angiography, traditional pulmonary angiography, any other now known or later developed imaging modality, or combinations thereof. Verification could also include the use of a two-dimensional imaging technique, a three-dimensional imaging technique, or combinations thereof.

If it is determined that the therapeutic treatments was not successful, the doctor 304, the soft tissue processor 108, or combinations thereof may then determine the need or desire to perform one or more therapeutic treatments (Block 522). Alternatively, where it is determined that the therapeutic treatment was successful, the patients may then be transferred from the patient support apparatus 116 to another surface, such as a hospital bed, or other medical equipment, such as a wheel-chair (Block 526). A successful determination may include the removal of a pulmonary embolism, the removal or diminishing of deep vein thrombosis, or combinations thereof. In another embodiment, if the therapeutic treatment was determined to be unsuccessful, the patient may then be transferred back to the hospital bed or other surface (Block 526). Transferring the patient from the patient support apparatus 116 may include the use of the soft tissue processor 108, the system controller 144, the motive device 142, the motive device 112, any other device or equipment, or combinations thereof. After the patient has been transferred from the patient support apparatus 116 to the hospital bed (Block 526), the patient is then returned to the emergency room or other department.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

We claim:

1. A system for performing phlebographic examinations, the system comprising:
a patient support apparatus operative to support a patient in a resting position;
a medical imaging device operative to acquire an image of a portion of the patient during a phlebographic examination, the medical imaging device being further operative to acquire the image using one of at least two imaging modalities; and,
a processor coupled with the medical imaging device operative to receive the acquired image of the patient, the processor being further operative to generate a visualization of the image of the portion of the patient.

2. The system of claim 1, wherein the patient support apparatus is further operative to rotate in a longitudinal direction about a horizontal axis.

3. The system of claim 1, wherein the patient support apparatus is further operative to rotate in a longitudinal direction about a vertical axis.

4. The system of claim 1, wherein the patient support apparatus is substantially transparent when exposed to radiation emitted from the medical imaging device.

5. The system of claim 1, wherein the patient support apparatus is rotatable about an axis of the medical imaging device.

6. The system of claim 1, wherein the patient support apparatus is mounted to a surface selected from the group consisting of a wall, a floor, and a ceiling.

7. The system of claim 1, wherein the medical imaging device is a C-Arm X-ray apparatus.

8. The system of claim 1, wherein the medical imaging device is rotatable around at least three axes.

9. The system of claim 1, wherein the medical imaging device is further operative to acquire the image of the portion of the patient using one of at least a two-dimensional imaging modality and a three-dimensional imaging technique.

10. The system of claim 1, wherein the processor is further operative to produce a two-dimensional visualization image of the portion of the patient acquired using the medical imaging device.

11. The system of claim 1, wherein the processor is further operative to produce a three-dimensional visualization of the image of the portion of the patient acquired using the medical imaging device.

12. The system of claim 1, wherein the processor is further operative to produce a four-dimensional visualization of the image of the portion of the patient acquired using the medical imaging device.

13. A method for performing a phlebographic examination, the method comprising:
providing a patient support apparatus operative to support a patient in a resting position;
providing a medical imaging device in a robotic angiographic suite, the medical imaging device operative to acquire an image of a portion of the patient during a phlebographic examination, the medical imaging device being further operative to acquire the image using one of at least two imaging modalities;
determining at least one phlebographic examination for performing on the patient; and,
performing the phlebographic examination on the patient positioned on the patient support apparatus using the medical imaging device.

14. The method of claim 13, further comprising:
determining whether the patient has a perceived difficulty in breathing; and,
adjusting the patient support apparatus to support the patient in a position that alleviates the difficulty the patient has in breathing.

15. The method of claim 13, wherein the phlebographic examination is a phlebography of the lower extremities of the patient.

16. The method of claim 13, wherein the phlebographic examination is a pulmonary angiography of the pulmonary arteries of the patient.

17. The method of claim 13, wherein the patient is diagnosed with a pulmonary embolism.

18. The method of claim 17, wherein the phlebographic examination comprises at least one of a phlebography of the lower extremities of the patient or a pulmonary angiography of the pulmonary arteries of the patient.

19. The method of claim 13, further comprising:
determining whether to perform a therapeutic treatment on the patient using the medical imaging device;
performing the therapeutic treatment on the patient using the medical imaging device; and,
verifying whether the therapeutic treatment was successful after the therapeutic treatment was performed.

20. The method of claim 19, wherein the therapeutic treatment is used in the treatment of at least one of a deep vein thrombosis or a pulmonary embolism.

21. The method of claim 13, wherein the patient support apparatus further operative to rotate in a longitudinal direction along a horizontal axis.

22. The method of claim 13, wherein the patient support apparatus is further operative to rotate in a longitudinal direction along a vertical axis.

23. The method of claim 13, wherein the medical imaging device is rotatable around at least three axes.

24. The method of claim 13, wherein the medical imaging device is further operative to acquire the image of the portion of the patient using one of at least a two-dimensional imaging modality and a three-dimensional imaging technique.
